(19) United States
(12) Patent Application Publication
(10) Pub. No.: US 2012/0205605 A1
(43) Pub. Date: Aug. 16, 2012

(54) ORGANIC REDOX ACTIVE COMPOUNDS WITH REVERSIBLE STORAGE OF CHARGES AND SUBSTRATES AND MOLECULAR MEMORY DEVICES COMPRISING THEM

(75) Inventors: Julien Buckley, Grenoble (FR); Renaud Demadrille, Saint-Egreve (FR); Kai Huang, Grenoble (FR); Yann Kervella, Montaud (FR)

(73) Assignee: Commissariat a' l' energie atomique et aux energie alternatives, Paris (FR)

(21) Appl. No.: 13/139,276

(22) PCT Filed: Dec. 9, 2009
(86) PCT No.: PCT/EP2009/066770

§ 371 Date: Jan. 30, 2012

(30) Foreign Application Priority Data
Dec. 11, 2008 (FR) 0858485

(51) Int. Cl. H01L 51/30; C07D 471/06

(52) U.S. Cl. 257/1; 546/66; 257/E51.025

(57) ABSTRACT

An organic redox active compound with reversible storage of charge is disclosed. The material characterized by a formula R-M-Y-T. According to some aspects, R represents a deconjugating group, M represents an organic redox active fragment, not comprising any metal ion or metal, capable of reversibly storing at least one charge, T represents a tripod group comprising three groups F, capable of being chemically grafted to a surface of a solid substrate, and Y represents a spacer group separating M from T. A substrate on which the compounds are grafted, a molecular memory device including the compound or the substrate, and an electronic apparatus including the molecular memory device are also disclosed.

### BACKGROUND

1. Field of the Invention

The present invention relates to organic redox active compounds with reversible storage of charges, in other words capable of storing charges reversibly.

The present invention further relates to a substrate and a molecular memory device comprising these organic redox active compounds as molecules capable of storing charges.

More specifically, this substrate is a substrate made of a semiconducting material, to a surface of which the organic redox active compounds with reversible storage of charges are chemically grafted.

The invention also relates to an electronic apparatus, especially a portable electronic apparatus comprising at least one such molecular memory device.

By memory device, is meant a device capable of receiving, retaining and/or restoring data.

The technical field of the invention may generally be defined as that of non-volatile molecular memories.

2. Description of the Prior Art

Memory devices consist of basic elements, memory cells, intended for storing data. Several types of memories exist i.e.:
random access memories, also called RAM (Random Access Memory) memories which are so-called volatile memories, since the data which they contain are lost after a few seconds, when the electric power is cut off and in which the data may be written, read or re-written as many times as necessary;
read-only memories, also called ROM (Read Only Memory) memories are memories programmed by the manufacturer and so-called non-volatile memories, since they retain the data which are contained therein in the absence of a power supply voltage, and in which the data are only read-accessible;
flash memories combine both the advantages of random access memories (in terms of writing, reading and deleting data blocks) and those of read-only memories (in terms of permanence of the contents even when it is not powered), the name of flash comes from the fact that the operations for deleting the memories are very fast, due to the fact that these memories are deletable per complete sector, and not per individual cell.

Herein, we will be more particularly interested in non-volatile memories, but the invention also applies to flash memories.

The technology of non-volatile memories based on semiconducting materials is presently confronted with two significant problems. The first relates to the increasing difficulty in reducing the size of these devices in order to reduce their unit cost and to increase the storage density. The second relates to the requirement of using high programming voltages which are generally greater than 10 V. The ideal operating voltages should be located below 1 V.

With molecular memories, which are hybrid electronic devices which use chemical molecules as a means for reversibly storing electric charges, it is possible de solve both of these problems.

The principle of these devices is based on the storage of charges at molecules which are generally metal complexes. In order to be able to store one or more charges, these metal complexes should have well defined redox properties and exist in at least two oxidation states or redox states. The molecule then has at least two states of charges, one of these states being the <<deleted>> state, and the other one being the <<written>> state. The switching from one state to the other is accomplished by transferring charges via an oxidation-reduction reaction mechanism, by applying a certain bias voltage to the molecule.

These molecular memories with charge storage have allowed reduction in the dimension of the devices and the use of lower supply voltages. Moreover, it has been demonstrated that the charge transfer rate may be adjusted by modulating the thickness of the layer, for example of silica, on which are grafted the molecules.

The use of molecular compounds, such as metallocenes and porphyrins, as a charge storage material has already been widely described in the literature.

Thus, document US-A1-2005/0121660 [1] describes hybrid devices with molecular memories comprising a film of active redox molecules which are especially porphyrins, metallocenes such as ferrocene, linear and cyclic polyenes, tetrathialfulvalenes, tetraselenafulvalenes, coordination complexes of metals, etc. The film may especially appear in the form of a self-assembled monolayer (or “SAM”).

As a reminder, “self-assembly” means the spontaneous formation of complex hierarchical structures from simple elements. The self-assembling phenomenon is at the basis of the formation of SAM, LBL or Langmuir-Blodgett layers. The forces involved in this phenomenon are of the supramolecular type, especially including Van der Waals, dipolar forces, hydrogen bonds.

Document U.S. Pat. No. 6,943,054 B2 [2] describes the coupling of an organic molecule, in particular a heat-resistant active redox molecule and provided with an anchoring group, with the surface of a semiconductor, by putting the molecule in contact with the surface and by heating the surface to a temperature of at least 200° C., in return for which the anchoring group forms a covalent bond with the surface.

Porphyrin monolayers on a silicon substrate (100) are especially prepared.

Document U.S. Pat. No. 7,324,385 B2 [3] relates to molecular memories comprising a thin layer of molecules for storing charges, which are macrocyclic complexes of metal ions. This thin layer may especially be in the form of a self-assembled monolayer (or SAM).

The anchoring groups giving the possibility of immobilizing the redox active fragment at the surface of the electrode may be bound to this surface via a single group—and this will then be referred to as a “monopod” immobilization or attachment—or via several groups—and this will then be referred to as a “polypod” or “multipod”, for example tripod immobilization or attachment—.

Document US-A1-2005/0243597 [4] relates to a device for making molecular memories in which a solution of the storage active molecules is applied to the surface of a substrate especially so as to form a self-assembled monolayer (or SAM).

The formation of the monolayers on silicon is based on the generation of a covalent bond of the Si—C type between the substrate and the molecule. The reaction allowing generation of this type of bond is known as hydrosilylation and it has been investigated in the literature very intensively.

However most of the articles, patents and patent applications which deal with the attachment, immobilization, fixing of molecules on silicon surfaces relate to molecules including a single anchoring function. In this respect, reference may be made to documents [1] to [4] already mentioned above as well as to the following documents: G. F. Cerofolini, G. Arena, C. M. Camalleri, C. Galati, S. Reina, L. Renna, D. Mascolo, “A hybrid approach to nanoelectronics”. Nanotechnology 16 (2005), 1040-1047 [5]; J. M. Buriak, “Organometallic chemistry on silicon surfaces: formation of functional monolayers bound through Si—C bonds” Chem. Commun. 1999, 1051-1060 [6]; T. Strother, R. J. Hamers, L. M. Smith, “Covalent attachment of oligodeoxyribonucleotides to amine-modified Si(001) surfaces”, Nucleic Acids Research, 2000, 28 (18), 3535-3541 [7].

The use of several identical anchoring functions for grafting redox active molecules on silicon surfaces has been reported recently.

Thus, the document of Z. Liu et al. <<Synthesis of porphyrins bearing hydrocarbon tethers and facile covalent attachment to Si(100)>>, J. Org. Chem. 2004, Vol. 69, 5568-5577 [8] describes the immobilization of 17 different porphyrins to Si(100) surfaces via carbosilane bonds. Among these porphyrins, three of them bear two identical (halogeno or vinyl) functional groups at two beta sites thereby allowing binding through two points to the surface of the silicon.

The document of K. Padmaja et al., <<A compact all carbon tripodal tether affords high coverage of porphyrins on silicon surfaces>>, J. Org. Chem. 2005, Vol. 70, No. 20, 7972-7978. [9] describes redox active molecules consisting in zinc, nickel and cobalt chelates of a molecule including as an anchoring group, a triallyl tripod connected via a p-phenylene unit to a porphyrin.

Monolayers of these molecules on surfaces of silicon (100) substrates have been prepared.

It was seen that multipod redox molecules are also described in document [3] already mentioned above.

The document of S. Katano, Y. Kim, H. Matsubara, T. Kitagawa, M. Kawai, <<Hierarchical chiral framework based on a rigid adamantane tripod on Au(111)>>”, J. Am. Chem. Soc. 2007, 129, 2511-2515 [10], indicates that the adsorption of bromo adamantane trithiol (BATT) molecules in the form of tripods on gold(111) leads to the formation of self-assembled monolayers (SAMs) with three contact points on the gold.

The main reasons which justify the use of multipod redox active molecules for grafting on the surfaces are especially the following:
by using several anchorings for one molecule it should be possible to obtain more robust layers in particular thermally;
the symmetry of the molecule gives better grafting density on the surface and leads to a better organized monolayer.

However, the multipod and especially tripod molecules mentioned in the literature in majority comprise metal ions, which has many drawbacks especially in terms of pollution, of high cost, and of mass.

Therefore, there exists a need for redox active molecules with charge storage which do not include metal ions.

These redox active molecules should further have electric stability, i.e. in other words, retention of the charge once it is stored, and stable redox states.

These redox active molecules should also have chemical stability, i.e. under the conditions of use, they should not undergo chemical reactions leading to their degradation or to the formation of undesirable byproducts.

These redox active molecules should finally have clearly distinct oxidation states as well as low charge transfer potentials.

The goal of the present invention is to provide organic redox active molecules, compounds, for reversible storage of charges, capable of reversibly storing charges, which i.a. meet the needs listed above, and which meet the aforementioned criteria and requirements.

The goal of the present invention is further to provide organic redox active molecules, compounds, for reversible storage of charges which do not have the drawbacks, defects, limitations and disadvantages of the organic redox active molecules, compounds, for storage of charges of the prior art, and which solve the problems posed by these compounds.

### DESCRIPTION OF CERTAIN INVENTIVE ASPECTS

This goal, and further other ones are attained, according to the invention by an organic redox active compound with reversible storage of charges of formula (I):

R-M-Y-T (I)

wherein:
R represents a deconjugating group;
M represents an organic redox active fragment not comprising any metal ion or metal, capable of reversibly storing at least one charge;
T represents a tripod group comprising three groups F, bound to a same atom and capable of being chemically grafted, capable of chemically grafting, preferably covalently, to a surface of a solid substrate;
Y represents a spacer group separating the organic redox active fragment M from the tripod group T.

The substrate may be made of a metal material, of an insulating material or of a semiconducting material, or of two or more of these materials. Accordingly, the surface of the substrate may be semiconducting, insulating or metallic.

The organic redox active compounds with reversible storage of charges of formula (I) according to the invention are novel compounds which have never been described in the prior art and which fundamentally differ from the organic redox active compounds of the prior art.

The compounds of formula (I) according to the invention have a totally novel specific structure comprising the four following essential elements R, M, T and Y at a time, associated in the same structure:
a tripod group T comprising three groups F, bound to a same atom, and capable of being chemically grafted, preferably covalently, to a surface of a substrate. These groups F may also be called anchoring functions or groups.

Because of the presence of this tripod group, the compound, the molecule according to the invention, has all the advantages typically associated with this type of groups mentioned earlier, as well as in document [9], especially in terms of robustness of the obtained layers, of stability, of high grafting density and therefore of reliable reading, and of better organization of the layer of molecules deposited on the substrate;
an optionally deconjugating spacer group Y which bears said tripod group T which separates and advantageously electrically insulates the organic redox active fragment M from the tripod group T and which allows control of the transfer of the charges from the substrate, for example made of silicon, towards the organic active redox fragment M;
an organic redox active fragment M which allows reversible storage of at least one charge. According to the invention, this redox active fragment is an organic fragment which does not comprise any metal entity capable of storing a charge, which is free of metal ion or metal.

Consequently, one of the main drawbacks of the compounds of the prior art such as porphyrins or metallocenes, which was specifically the presence of metal ions and of metals, is overcome.

The compounds according to the invention are entirely organic compounds without any metal entities capable of storing a charge, free of metal ions (in their structure) or of metals, many advantages ensue, among which mention may be made of the reduction of pollution by metal salts or by metals during the manufacturing of molecular memory devices and during their disposal as well, of the cost reduction of these devices, and of the reduction in the total mass of the devices.
a deconjugating group R with which it is possible to monitor, modulate, control the charge injection into the organic redox active fragment M, and therefore to monitor, modulate, control the charge transfer potentials between the fragment M and the substrate.

The group R may easily be modified, modulated, this is preferably an electrically insulating group.

By varying the length of this group consisting for example of an alkyl chain and/or its size, it is possible to easily control the injection of charges. A priori, the longer and bulkier the chain, the more it will be insulating and the more the injection of charges will be difficult.

Surprisingly, the inventors have associated in a same molecule a deconjugating group, preferably an electrically insulating group R and an organic redox active fragment M, which ensures control of the injection of the charges.

On the other hand, the spacer group Y allows control of the transfer of charges from the substrate. The circulation of the charges from and towards the fragment M and from and towards the substrate is therefore perfectly under control in the compounds according to the invention.

Further, in a totally surprising way, the compounds according to the invention in their portion dedicated to charge storage (M) do not include any metal entity capable of storing a charge, do not comprise any metal ions or metals but however have excellent electrical and chemical stability properties. The compounds according to the invention meet the requirements and criteria mentioned above.

Further, the oxidation states of the compounds according to the invention are clearly distinct.

The charge transfer potential of the compounds according to the invention is low, for example from −5 to +5 volts and, as this has already been mentioned earlier, may even be further reduced for example down to −2 volts in the case of long and/or bulky groups R, especially groups R containing carbon chains, in particular long carbon chains for example from 6 to 16 carbon atoms, especially from 8 to 12 carbon atoms.

Accordingly, the use of redox molecules according to the invention in memory devices allows considerable reduction in the power consumption of these memory devices.

As a conclusion, the compound according to the invention inter alia meet the needs listed above, solve the problems which were exhibited by the compounds of the prior art, and do not exhibit the drawbacks of these compounds of the prior art while having all the advantages of these compounds.

Before describing the invention in a more detailed way, we specify the following definitions:
By substrate made of a metal material is meant a substrate made of a solid material selected from metals, metal alloys known to the man skilled in the art, such as noble metals, especially Ag, Au, Pt, Pd, transition metals like Cu or Ni, and alloys comprising these metals;
By substrate made of an insulating material is meant a substrate made of a solid material selected from insulating materials like oxides such as SiO₂, TiO₂, ZrO;
By substrate made of a semiconducting material, is meant a substrate made of a solid material selected from semiconducting materials known to the man skilled in the art, such as silicon, germanium, SiGe, ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe.

The preferred semiconducting materials are silicon, germanium, gallium and their derivatives, optionally doped for example with boron or with phosphorus.

The crystalline orientation of the substrate may vary. Thus, in the case of silicon, the latter may have a (100) or (111) crystalline orientation, whether it is intrinsic or doped.

The substrate may be made of a single material or else of a mixture of two or more materials.

Although this is not always necessary, the surface of the substrate, in particular made of silicon may be cleaned before its use, before grafting the compounds according to the invention by applying standard cleaning and/or pickling procedures.

Thus for example, the surface, especially made of silicon, may be cleaned in one or more solvents from the following solvents used simultaneously or successively: acetone, toluene, ethanol and water, and then pickled with a standard solution for cleaning wafers such as a mixture of sulfuric acid and of hydrogen peroxide for example in volume proportions of 3/1. For example, it is possible to immerse the surface for a period of generally 1 to 5 minutes in this mixture, and then to abundantly rinse it with deionized water and dry it for example under a flow of argon.

By treatment with this mixture of H₂SO₄ and H₂O₂, the surface, for example made of silicon, may be pickled and any trace of organic residue may be removed before the grafting.

In certain cases and especially in the case of silicon, oxides may be removed from the surface of the substrate and the surface may then be hydrogenated (passivated with hydrogen).

Indeed, in the case of silicon surfaces, a native oxide SiO₂ layer, generally of small thickness, for example of about 2 nm, is spontaneously formed in air and it is recommended to remove it before grafting the compounds according to the invention.

This oxide layer may be removed by pickling for example with a 1% hydrofluoric acid solution. This surface may thus be immersed in the HF solution for a period for example of 1 minute, and then dried.

The hydrogenation of the surface, in other words its passivation by hydrogen, may be accomplished by any common method known to the man skilled in the art, for example by putting the surface to be passivated into contact with an ammonium fluoride solution.

It is also specified that this substrate may appear in any shape, for example as a block, (or a part), or as a coating, for example with a thickness from 10 nm to 100 μm.

The surface to which the compounds of formula (I) according to the invention are grafted, is preferably a planar surface.
By <<redox active>> or <<active redox>> compound, molecule or fragment is generally meant that this compound, this molecule or this fragment may be oxidized or reduced by applying an adequate voltage to them;
By electrically insulating group, is generally meant that this group totally or partly prevents or limits the transfer, the transport of electric charges and quantitatively, regulates, modulates, controls this transport, transfer ;
By deconjugating group, is generally meant that this group achieves breakage of the conjugation by breaking the overlapping of pi orbitals;
By tripod group in the sense of the invention, is meant that this group comprises three groups F, bound to one single and same atom such as a carbon or silicon atom, these three groups F being preferably identical, and being preferably allyl groups;
   By chemical grafting is meant in the foregoing and in the following, immobilization of the aforementioned compound(s) of formula (I) on the substrate by means of an advantageously covalent, or even ionocovalent, chemical bond. It is specified that this immobilization is accomplished at the surface of the substrate.

It is well understood that chemical grafting does not exclude the existence of simple physical interactions such as the so-called Van der Waals interactions or interactions of the hydrogen bond type between the aforementioned compounds of formula (I) and the aforementioned substrate.
By group F capable of being chemically grafted to said substrate are meant groups reacting with the reactive groups present at the surface of the substrate, advantageously made of a semiconducting material, such as silane groups Si—H in the case of a hydrogenated silicon substrate.
By spacer group, is generally meant a unit consisting in at least one atom, separating two functional entities.

The group R represents a deconjugating group, preferably an electrically insulating group.

Advantageously, the group R is selected from hydrogen, alkyl groups, alkoxy groups; heterocycles; aryl groups.

Preferably the group R is an n-octyl group or a n-dodecyl group.

In the present description, the term “alkyl” used for alkyl radicals, groups, as well as for groups, radicals, fragment groups, including an alkyl portion, means, unless otherwise mentioned, a saturated linear or branched or cyclic carbon chain, including from 1 to 30 carbon atoms, preferably from 1 to 24 carbon atoms, still preferably from 1 to 16, better from 1 to 8, still better from 1 to 4 carbon atoms, which may be optionally substituted with one or more identical or different group(s), generally selected from halogen atoms such as chlorine, bromine, iodine and fluorine; heterocycles; optionally substituted aryl radicals; hydroxyl; alkoxy; amino; C₂-C₈ acyl; carboxamido; CO₂H; alkoxycarboxyl, carboxyamino, —SO₃H; —PO₃H₂; —PO₄H₂; —NHSO₃H; sulfonamide; cyano; monoalkylamino; trialkylammonium radicals; or else further with a dialkylamino radical.

In the case when the alkyl radical is a linear or branched radical, one or more carbon atoms of the radical may be replaced with one or more carbonyl group(s) and/or one or more hetero-atom(s) selected from nitrogen, oxygen and sulfur atoms.

In the case when the alkyl radical is a cyclic radical, it generally has from 3 to 30 carbon atoms, preferably from 4 to 16 carbon atoms, better from 4 to 8 carbon atoms, still better from 5 to 7 carbon atoms and does not comprise any carbon-carbon double bond, and one or more carbon atoms of the cyclic radical may be replaced with one or more carbonyl groups.

Also, according to the invention, the term “alkoxy” used for alkoxy radicals as well as for groups including an alkoxy portion, means, unless otherwise mentioned, an O-alkyl chain, the term “alkyl” having the meaning indicated above.

According to the invention, by heterocycle is meant a cycle, either saturated or not, either aromatic or not, containing from 5 to 12 members, preferably from 5 to 7 members, and preferably from 1 to 3 hetero-atoms selected from nitrogen, sulfur and oxygen atoms. These heterocycles may be fused on other heterocycles, or on other, especially aromatic, cycles such as a phenyl group. These heterocycles may further be quaternized especially with an alkyl radical.

Among the heterocycles, either fused or not, mention may especially be made as an example of the cycles: thiophene, benzothiophene, furane, benzofurane, indole, indoline, carbazole, pyridine, dehydroquinoline, chromone, julodinine, thiadiazole, triazole, isoxazole, oxazole, thiazole, isothiazole, imidazole, pyrrazole, triazine, thiazine, pyrazine, pyridazine, pyrimidine, diazepine, oxazepine, benzotriazole, benzoxazole, benzimidazole, benzothiazole, morpholine, piperidine, piperazine, azetidine, pyrrolidine, aziridine, pyrrole, piperidine.

According to the invention, it is specified that a heterocycle may optionally be substituted. In this case it bears one or more substituents, either identical or not, generally selected from optionally substituted, linear or branched C₁-C₁₆ preferably C₁-C₁₀ alkyl radicals, carboxy radicals, linear or branched, alkoxycarbonyl radicals, the alkoxy of which is a C₁-C₁₆ alkoxy, preferably a C₁-C₁₀ alkoxy, amino radicals, amino-alkyl or aminoalkyl carbamoyl (H₂N-alkyl-NH—CO—) radicals, in which the alkyl portion is a linear or branched C₁-C₁₆, preferably C₁-C₁₀alkyl.

According to the invention one or more of the carbon atoms of the heterocyclic groups may be replaced with one or more carbonyl groups.

According to the invention, is meant by aryl unless otherwise mentioned, a cyclic C₆-C₃₀ conjugate system having aromaticity, which may be substituted with one or more groups either identical or different, selected from halogen atoms; C₁-C₁₆, preferably C₁-C₁₀ linear or branched alkyl radicals; C₁-C₁₆ linear or branched alkoxy radicals, preferably C₁-C₁₀ linear or branched alkoxy radicals; optionally substituted aryloxy radicals; mesyl (CH₃—SO₂—); cyano; carboxamido; —CO₂H; sulfo (SO₃H); —PO₃H₂; —PO₄H₂; hydroxyl; amino radicals; mono or di-substituted amino radical such as mono-(C₁-C₄)alkylamino or di-(C₁-C₄) alkylamino radicals.

By aryl is also meant in the sense of the invention, a radical, especially a divalent radical such as a biphenyl radical comprising several aryl radicals as defined above bound through a simple bond or an alkyl chain.

Preferably, the aryl group is a phenyl group or a naphthyl group which may be substituted as indicated above.

The organic redox active fragment M is advantageously selected from fragments comprising one or more polycyclic group(s) with pi conjugation such as naphthalene, phenanthrene, anthracene, tetracene and coronene.

The fragment M should allow reversible storage of at least one charge, but advantageously the redox system of this fragment should have multiple and accessible redox states and allow storage of several charges, for example from 2 to 4 charges.

Preferably, the organic redox active fragment M fits the following formula (IIA), (IIB), (IIC), (IID) or (IIIE):

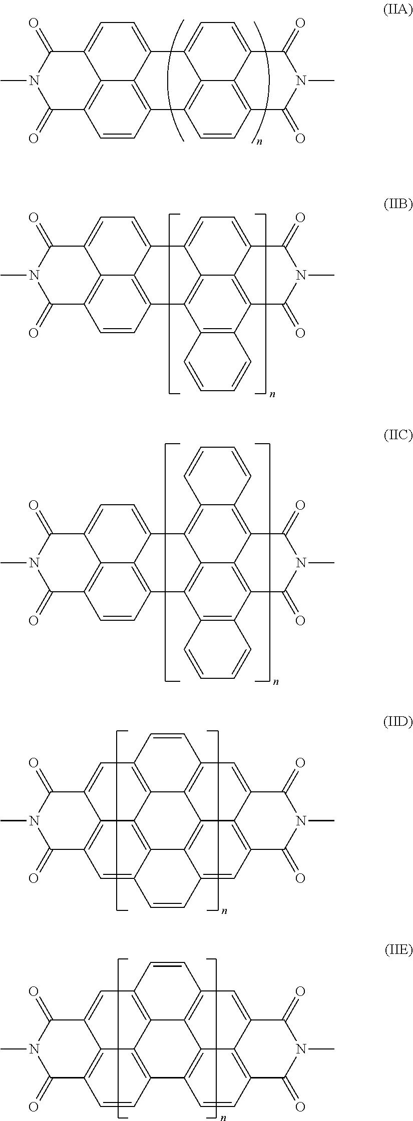

wherein n is an integer which may assume all the values from 0 to 6, such as 0, 1, 2, 3.

In the case when n is equal to 0 in formula (IIA), the fragment M is a naphthalene tetracarboxydimide fragment.

In the formulae (IIB) and (IIC), the naphthalene between square brackets in formula (IA) is replaced with anthracene and tetracene respectively.

In the formulae (IID) and (IIE), the organic redox active fragment M is based on coronene and phenanthrene.

The fragment M may further be optionally substituted with one or more electron donor or electron acceptor group(s) in order to modulate the oxidation and reduction levels.

The electron donor group(s) may especially be selected from alkyl groups, alkoxy groups, amines.

The electron acceptor group(s) may especially be selected from halogens, carbonyls, the nitrile function.

Advantageously, the spacer group Y is electrically insulating, it is generally selected from divalent groups corresponding to the monovalent groups mentioned for the group R, i.e. divalent alkyl groups (alkylene); divalent alkoxy groups; divalent (aromatic or non-aromatic) heterocycles; and divalent aryl groups.

Preferred groups for Y are the phenylene group and the diphenylene group.

Advantageously, the groups F capable of being chemically bound, preferably covalently to a surface of a substrate made of a material, preferably made of a semiconducting material are typically selected from the functions, groups: hydroxyl, mercapto, selenyl, telluryl, cyano, isocyano, carboxyl, amino, dihydroxyphosphoryl, dithio, dithiocarboxyl, diazonium, halogeno, alkenyl such as allyl, alkynyl, alkoxyl, silyl, phosphate-phosphonate; and carbon chains preferably from 1 to 5 carbon atoms, advantageously with 3 carbon atoms, including one or more functions, groups selected from functions, groups: hydroxyl, mercapto, selenyl, telluryl, cyano, isocyano, carboxyl, amino, dihydroxyphosphoryl, dithio, dithiocarboxyl, diazonium, halogeno, alkenyl such as allyl, alkynyl, alkoxyl, silyl, phosphate-phosphonate.

The preferred group F is the allyl group.

Advantageously, the tripod group is a 4-allylhepta-1,6-dien-4-yl group of formula —C—(—CH₂—CH═CH₂)₃, or a tripod group of formula Si—(—CH₂—CH═CH₂)₃.

The compound according to the invention may advantageously fit the following formula (IIIA), (IIIB), (IIIC), (IIID), or (IIIE):

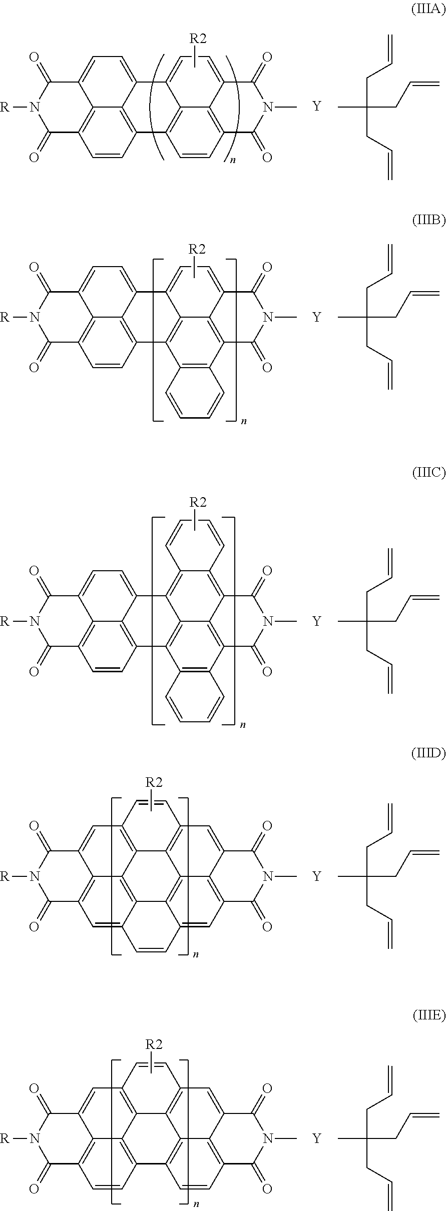

wherein R, n and Y have already been defined above and R2 represents a hydrogen or an electron donor or electron attractor group.

Preferred compounds according to the invention are the following compounds:
N-[4-(4-allylhepta-1,6-dien-4-yl)phenyl]-1,4,5,8-naphthalenetetracarboxydiimide;
N-[4-(4-allylhepta-1,6-dien-4-yl)phenyl]-N′-n-octyl-1,4,5,8-naphthalenetetracarboxydiimide
N-[4-(4-allylhepta-1,6-dien-4-yl)phenyl]-N′-n-dodecyl-1,4,5,8-naphthalenetetracarboxydiimide.

The structural formulae of these compounds are given hereafter:

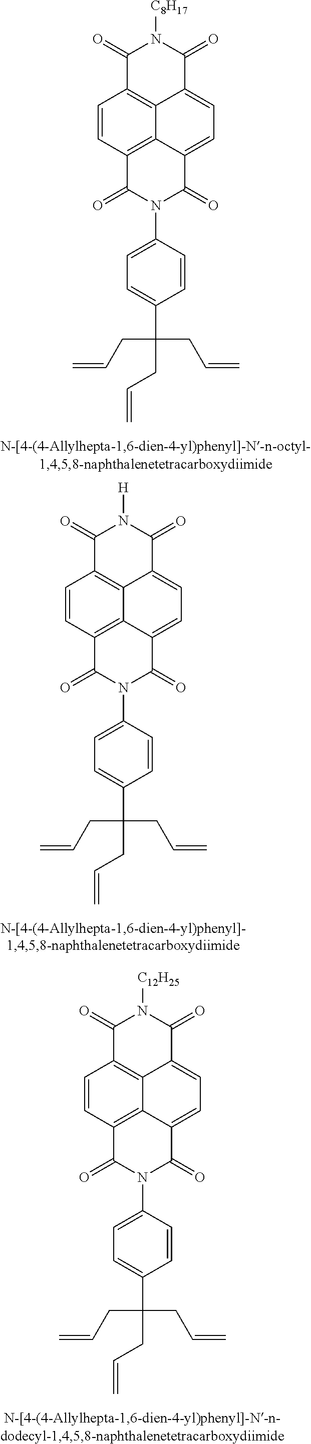

The compounds according to the invention may generally be prepared by coupling from a first precursor, including a redox active fragment as defined beforehand as well as a deconjugating group R, and from a second precursor including a tripod group, which may be bound beforehand to the substrate, as well as a spacer group Y, as defined beforehand. These precursors are commonly called precursors of the compound (I).

The first precursor or entity and the second precursor or entity advantageously include additional functions which may lead to the formation of a covalent bond between both precursors, both entities, when they are placed under suitable conditions, preferentially mild and non-destabilizing conditions for the other functions present on the precursors, entities.

If necessary, it is of course possible to protect the most sensitive organic functions according to the most common methods of organic chemistry.

As regards the protection of organic functions, it is possible to refer to the following text book: <<Protective Groups in Organic Synthesis>>, Theodora W. Greene, Peter G. M. Wuts, ed. Wiley-Interscience, 3^{rd} edition, 1999 [11].

It is preferable that the functions allowing the coupling, be localized on the redox fragment for the first precursor and on the spacer group Y for the second precursor.

As an example of a coupling reaction, it is for example possible to mention condensation reactions, which may for example lead to ester or imide bonds. Indeed, the latter is typically achieved simply by heating. As such, reference may especially be made to the examples.

Of course it is possible to use other coupling reactions, the latter being adapted depending on the type of precursor available to the man skilled in the art.

The invention also relates to a substrate, preferably made of a semiconducting material, to a surface of which are chemically, preferably covalently, grafted organic redox active compounds capable of storing charges of formula (I) according to the invention as defined above.

This substrate may be defined as a substrate made of an organic (the compound of formula (I))/inorganic (the solid material of the substrate, for example the semiconducting, insulating, or metal, material of the substrate) hybrid material.

This material is novel and has advantageous properties, essentially due to the compound of formula (I), which properties have already been described above.

The substrate and the material preferably the semiconducting material, making it up have already been defined above.

Advantageously, this semiconducting material is silicon, optionally doped.

As a reminder, the substrate, as this was mentioned above may be a substrate having optionally undergone cleaning and/or pickling and/or surface oxides removal, and hydrogenation treatments.

In order to obtain the grafting of the compound of formula (I) according to the invention to/on a surface of the substrate, preferably a semiconducting substrate, different techniques may be contemplated, in particular techniques via a liquid route i.e. techniques for impregnating the aforementioned substrate with an organic solution comprising the compound(s) of formula (I) or else its (their) precursors as defined above.

Thus, the chemical grafting at the surface of the substrate, preferably made of a semiconducting material, may be carried out with one of the following impregnation techniques:
dip-coating;
spin-coating;
laminar-flow-coating;
spray-coating;
soak-coating;
roll-to-roll process
painting coating;
screen-printing.

This operation is generally followed by a step allowing the reaction between the surface, for example made of silicon and the tripod, for example a tripod group including F groups of the allyl type. As indicated in the examples, this may be a step of heating around 180° C. for example, with which covalent bonds may be generated between the molecules and the surface.

The reaction applied in this step may be of diverse nature and may especially be photo assisted.

According to a particular mode, the step for preparing the compounds (I) from its precursors, by coupling, and the grafting reaction between the surface of the substrate and the tripod group are performed at the same time.

In this case it is preferable that the applied coupling and grafting conditions be compatible.

These different techniques (impregnation and then reaction) are generally applied for a suitable period of time, in order to allow optimum contact of the substrate with the organic solution comprising the compound(s) (I) or its(their) precursor(s), so that the substrate is totally impregnated on its surface and the compounds may react at the surface. For example, this period of time may be from 1 to 48 hours, for example 16 hours.

The reaction may be tracked, monitored, by suitable spectroscopic means such as infrared spectroscopy or X-ray photoelectron spectrometry (XPS).

The solvent of said solution may be easily selected by the man skilled in the art.

This solvent may for example be selected from THF, from C₁-C₈ aliphatic alcohols such as methanol and ethanol, halogenated solvents, aromatic solvents, and mixtures thereof.

The concentration of the compound (I) or of its precursors in said solution may easily be determined by the man skilled in the art, it is generally from 10⁻³ to 1 M.

When the temperature at which the impregnation is performed may also be easily determined by the man skilled in the art, it is generally from 20° C. to 80° C., preferably this impregnation is performed at room temperature.

A preferred method for achieving grafting of the molecules (I) according to the invention especially in the case of a silicon surface cleaned, pickled beforehand, the native oxide layer of which has been removed, and which was hydrogenated as described above, is a method applying thermal hydrosilylation.

Surfaces of silicon with different crystallinities may be used.

Such a method is notably described in the document of A. B. Sieval, A. L. Demirel, J. W. M. Nissink, M. R. Linford J. H. van Der Mass, W. H. de Jeu, H. Zuilhof, E. J. R. Sudholter, <<Highly stable Si—C linked functionalized monolayers on the silicon (100) surface>> Langmuir, 1998, 14(7), 1759 [12], to the description of which reference may be made.

Thermal hydrosilylation may thus be performed by placing the freshly pickled and hydrogenated silicon substrates in a solution of mesitylene containing the molecule of formula (I) according to the invention and then by reflux heating for example for 2 hours. The reaction is generally conducted under an inert atmosphere, for example an argon atmosphere.

After this grafting or functionalization step, the method for preparing the substrate made of an inorganic-organic hybrid material according to the invention may comprise a treatment step intended to remove the residues of the grafting reaction as well as the unreacted species.

This treatment may consist in rinsing the hybrid material with an organic solvent which preferably is the same solvent as the one used for the grafting.

Finally, it is generally proceeded with drying of the substrate made of an inorganic-organic hybrid material.

The compounds according to the invention may form a monolayer at the surface of the substrate, for example with a thickness from 2 to 3 nm.

These monolayers, because of the specific structure of the compounds according to the invention, are robust, especially thermally, and better organized than the layers prepared by grafting of the compounds of the prior art.

The present invention also relates to a molecular memory device comprising an organic redox active compound with reversible storage of charges of formula (I) according to the invention, or comprising a substrate, advantageously made of a semiconducting material, to a surface of which is chemically grafted, advantageously covalently, a redox active compound with reversible storage of charges of formula (I) according to the invention, as defined above.

The substrate is advantageously an electrode of the molecular memory device and preferably a working electrode.

Such a device is known to the man skilled in the art and will not be described in more detail herein. It only differs from known devices by the application of the specific compounds according to the invention.

Such a device and its manufacturing are for example described in document US-A1-2003/0111670 [13].

The device according to the invention has all the advantages already mentioned above, and due to the use of the molecules of formula (I) according to the invention.

The invention also relates to an electronic apparatus comprising at least one memory device as defined above, the apparatus may be selected from portable electronic apparatuses, such as digital still cameras, cellphones, printers, portable computers or sound playing and recording devices such as personal music MP3 players.

Finally, the invention relates to the use of the compounds of formula (I) as molecules able to store charges in a molecular memory device.

The present invention will now be described in connection with exemplary embodiments, given as an illustration and not as a limitation.

### BRIEF DESCRIPTION OF THE DRAWINGS

FIG. 1A is the X-ray photoelectron spectroscopy (XPS) spectrum of the molecule 1 according to the invention, wherein R is a hydrogen, prepared in Example 1, grafted on a hydrogenated silicon (100) substrate. This spectrum gives the number of emitted electrons versus the binding energy of the electrons (in eV);

FIGS. 1B, 1C, 1D and 1E are enlargements of portions of the spectrum of FIG. 1A which respectively relate to the peaks involving carbon, nitrogen, silicon and oxygen atoms of the molecule 1;

FIG. 2A is the X-ray photoelectron spectroscopy (XPS) spectrum of the molecule 2 according to the invention, wherein R is an n-octyl group, prepared in Example 2, grafted on a hydrogenated silicon (100) substrate. This spectrum gives the number of emitted electrons versus the binding energy of the electrons (in eV);

FIGS. 2B, 2C and 2D are enlargements of portions of the spectrum of FIG. 2A which respectively relate to the peaks involving silicon, nitrogen and carbon atoms of the molecule 2;

FIG. 3 is a schematic vertical sectional view of the measurement device used for determining the electrical properties of the molecules 1, 2, and 3 according to the invention, prepared in Examples 1, 2 and 3;

FIG. 4A is a graph which gives the capacitance C (in F/cm²) versus the gate voltage VG (in volts) for a monolayer of the molecules 1 according to the invention, grafted on a silicon substrate, and integrated into the device of FIG. 3; the upper curve (□) was established at 70 Hz and the lower curve (◯) was established at 500 Hz;

FIG. 4B is a graph which gives the conductance G (in S/cm²) versus the gate voltage VG (in volts) for a monolayer of the molecules 1 according to the invention, grafted on a silicon substrate, and integrated into the device of FIG. 3; the upper curve (□) was established at 70 Hz and the lower layer (◯) was established at 500 Hz;

FIG. 5A is a graph which gives the capacitance C (in F/cm²) versus the gate voltage VG (in volts) for a monolayer of the molecules 2 according to the invention grafted on a silicon substrate, and integrated into the device of FIG. 3; the upper curve (◯) was established at 70 Hz and the lower layer (□) was established at 500 Hz;

FIG. 5B is a graph which gives the conductance G (in S/cm²) versus the gate voltage VG (in volts) for a monolayer of the molecules 2 according to the invention, grafted on a silicon substrate, and integrated into the device of FIG. 3; the upper curve (□) was established at 70 Hz and the lower layer (◯) was established at 500 Hz.

FIG. 6A is a graph which gives the capacitance C (in F/cm²) versus the gate voltage VG (in volts) for a monolayer of the molecules 3 according to the invention, grafted on a silicon substrate, and integrated into the device of FIG. 3; the upper curve (◯) was established at 70 Hz and the lower layer (□) was established at 500 Hz.

FIG. 6B is a graph which gives the conductance G (in S/cm²) versus the gate voltage VG (in volts) for a monolayer of the molecules 3 according to the invention, grafted on a silicon substrate, and integrated into the device of FIG. 3; the upper curve (□) was established at 70 Hz and the lower layer (◯) was established at 500 Hz.

### DETAILED DISCUSSION OF CERTAIN ILLUSTRATIVE EMBODIMENTS

### Example 1

### Synthesis of N-[4-(4-allylhepta-1,6-dien-4-yl)phenyl]-1,4,5,8-naphthalenetetracarboxydiimide (Molecule 1)

In this example, the synthesis of N-[4-(4-allylhepta-1,6-dien-4-yl)phenyl]-1,4,5,8-naphthalenetetracarboxydiimide is carried out in two steps. (Molecule 1 according to the invention).

### Step 1: Synthesis of N-[4-(4-allylhepta-1,6-dien-4-yl)phenyl]-1,4,5,8-naphthalenetetracarboxyanhydride

In a two-neck round-bottom flask equipped with a dropping funnel and of a condenser of the Dean-Starck type, 1,4,5,8-naphthalene-tetracarboxydianhydride (1 equiv. 5.9 g, 22 mmol) is dissolved in 100 mL of anhydrous DMF at 160° C. 4-(4-allylhepta-1,6-dien-4-yl) aniline (1 equiv. 5 g, 22 mmol) dissolved beforehand in 50 mL of anhydrous DMF is added dropwise to the anhydride solution. The reaction is refluxed for 12 hrs. At the end of the reflux, the DMF is evaporated in vacuo and the product is then purified with a silica gel chromatography column with a mixture of ethyl acetate and dichloromethane (1/1 v/v). A beige powder is obtained which is washed with methanol. After filtration and drying, 7.9 g are obtained (yield: 75.7%).

### Step 2: Synthesis of N-[4-(4-allylhepta-1,6-dien-4-yl)phenyl]-1,4,5,8-naphthalene-tetracarboxydiimide (Molecule 1)

The N-[4-(4-allylhepta-1,6-dien-4-yl)-phenyl]-1,4,5,8-naphthalenetetracarboxyanhydride (1 equiv. 7.07 g, 14.73 mmol) prepared in Example 1 and ammonium acetate (20 equiv., 21.56 g. 280 mmol) are dissolved in 100 mL of acetic acid and 100 mL of chloroform. The reaction is refluxed at 120° C. for 4 hours. After returning to room temperature, the chloroform is evaporated in vacuo and a precipitate is formed. After filtration, a solid is obtained which is washed with water, with methanol and with ethyl ether. Next, the solid is dried and a pale beige powder of the molecule 1 according to the invention is obtained (5.43 g, yield: 76.7%).

¹H-NMR (DMSO-d6, 200 MHz, ppm): δ 12.15 (s, 1H, NH), 8.63 (s, 4H), 7.52 (d, 2H, J=7.34 Hz), 7.36 (d, 2H, J=7.34 Hz); 5.55-5.43 (m, 3H); 5.02-4.95 (m, 6H), 2.60-2.38 (m, 6H).

### Example 2

### Synthesis of molecule 2 (with R=n-octyl). N-octyl-N-[4-(4-allylhepta-1,6-dien-4-yl)phenyl]-1,4,5,8-naphthalene tetracarboxydiimide

In a two-neck round-bottom flask equipped with a dropping funnel and a condenser of the Dean-Starck type, N-octyl-1,8-naphthalene-dicarboximide 4,5-dicarboxyanhydride, (1 equiv., 1 g, 2.64 mmol) is dissolved in 50 mL of anhydrous DMF at 160° C. 4-(4-allylhepta-1,6-dien-4-yl) aniline (1 equiv., 0.6 g, 2.64 mmol) dissolved beforehand in 10 mL of anhydrous DMF is added dropwise to the anhydride solution. The reaction is refluxed for 15 hrs. At the end of the reflux, the DMF is evaporated in vacuo and the product is then purified with a silica gel chromatography column with a mixture of ethyl acetate and dichloromethane (1/1 v/v). A pink powder is obtained which is washed with methanol. (1.13 g, yield: 73%).

¹H-NMR (CHCl₃-d, 62.5 MHz, ppm): δ 14.57 (CH₃) 23.10 (CH₂), 27.55 (CH₂), 28.54 (CH₂), 29.66 (CH₂) 29.75 (CH₂), 32.26 (CH₂), 41.52 (Cq), 42.32 (CH₂) 43.89 (CH₂), 118.43 (3=CH₂), 127.19 (2CH), 127.26 (2Cq) 127.36 (CH), 127.49 (Cq), 128.26 (2C), 128.39 (2C), 131.50 (2CH), 131.75 (2CH), 132.55 (C), 134.69 (3CH) 147.29 (Cq), 163.22 (2C═O), 163.48 (2C═O).

### Example 3

### Synthesis of Molecule 3 (with R=n-dodecyl). N-dodecyl-N-[4-(4-allylhepta-1,6-dien-4-yl)phenyl]-1,4,5,8-naphthalene tetracarboxy diimide

In a two-neck round-bottom flask equipped with a dropping funnel and a condenser of the Dean-Starck type N-dodecyl-1,8-naphthalene dicarboximide 4,5-dicarboxyanhydride, (1 equiv.) is dissolved in 50 mL of anhydrous DMF at 160° C. 4-(4-allylhepta-1,6-dien-4-yl) aniline (1 equiv.) dissolved beforehand in 10 mL of anhydrous DMF is added dropwise to the anhydride solution. The reaction is refluxed for 14 h. At the end of the reflux the DMF is evaporated in vacuo and the product is then purified with a silica gel chromatography column with a mixture of ethyl acetate and dichloromethane (1/1 v/v). A pinkish powder is obtained which is washed with methanol.

### Example 4

### Grafting the molecules 1, 2 and 3 on a Silicon Substrate

In this example, the grafting of the molecules 1, 2 and 3 according to the invention is accomplished by a thermal hydrosilylation reaction on a surface of hydrogenated silicon. For more details on this hydrosilylation reaction, reference may be made to document [12], already mentioned.

The crystalline orientation of the substrate is Si(100). This is a P type conducting substrate, 7-10 Ω·cm, doped with boron, the size of the active surface area is 150×300 μm² and 100×790 μm².

In order to obtain a hydrogenated silicon surface of good quality, the native oxide layer which is spontaneously formed in air has to be pickled.

In order to pickle the surface and to remove any trace of organic residue before the grafting, the device is first of all pickled with a mixture of concentrated H₂SO₄ and of H₂O₂ (v/v 3/1). The device is immersed for 1 to 5 minutes in this solution and abundantly rinsed with deionized water and then dried under a flow of argon.

Next, only the portion consisting of native oxide of thin thickness is pickled by immersion into a 1% HF solution for 1 min and then dried.

It is then proceeded with hydrogenation by immersion in 1% HF for 5 minutes of the surface which has been pickled with hydrofluoric acid.

The formation of the Si—H functions is tracked by X-ray photoelectron spectrometry (XPS) and infrared spectrometry.

The grafting of the molecules is accomplished as already specified by a thermal hydrosilylation reaction by placing the freshly pickled silicon substrate in a solution of mesitylene containing the molecule and then by refluxing for 2 hr with heating. The reaction is conducted under an inert atmosphere.

The grafted surfaces are characterized by X-ray photoelectron spectrometry (XPS).

The XPS measurements are made with an S-Probe spectrometer using a monochromatic line Kα (1486.6 eV photons) with a dwell time of 100 ms and an energy pass of 50 eV.

The signal is obtained at a take-off angle α (measured relatively to the surface) of 35°.

The pressure in the analysis chamber is 10⁻⁹ Torrs or less at each measurement.

The reference used for the binding energies is the Au 4f peak at 84 eV.

The signal C is at 284.6 eV shows that no charge effect is observed.

The photoelectrons are detected by using a hemispherical analysis sphere, with an angular limit of 30° and an energy resolution of 850 meV.

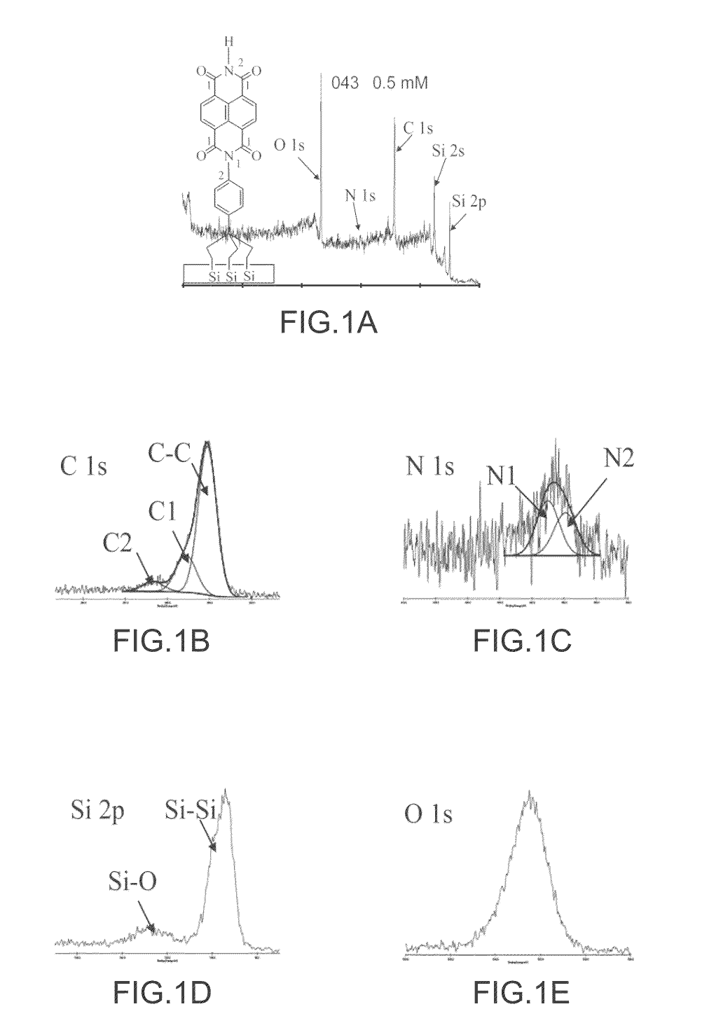

FIG. 1A gives the X-ray photoelectron spectrophotometry spectrum (XPS) of the molecule 1 according to the invention, grafted on a hydrogenated silicon (100) substrate, and FIGS. 1B to 1E are enlargements of portions of the spectrum of FIG. 1A.

In FIG. 1A, the peaks corresponding to O (1s), N (1s), C (1s), Si (2s), Si (2p) have been indicated.

In FIG. 1B, the peaks corresponding to the carbons C1 and C2 of the molecule 1 and to the C—C bonds of the benzene rings have been identified.

In FIG. 1C, the peaks corresponding to the nitrogen atoms N1, N2 of the molecule 1 have been identified.

In FIG. 1D, the peaks corresponding to the Si—O and Si—Si bonds have been identified.

In FIG. 1E, the peak corresponding to the oxygen atoms of the carbonyl groups has been identified.

Table 1 gives the numerical values of the parameters describing the spectrum of FIG. 1A.

TABLE 1

| XPS peak | Adjusted | | | |
|---|---|---|---|---|
| of the | Binding | Normalized | Sensitivity | Atomic |
| element | Energy | Area | (*) | % |
| Si 2p | 100.032 | 18.558 | 0.84 | 30.829 |
| O 1s | 532.636 | 11.983 | 2.80 | 19.907 |
| C 1s | 285.770 | 28.117 | 1.00 | 46.809 |
| N 1s | 400.625 | 1.478 | 1.76 | 2.455 |

(*) the sensitivity is the sensitivity of the detector to the relevant element.

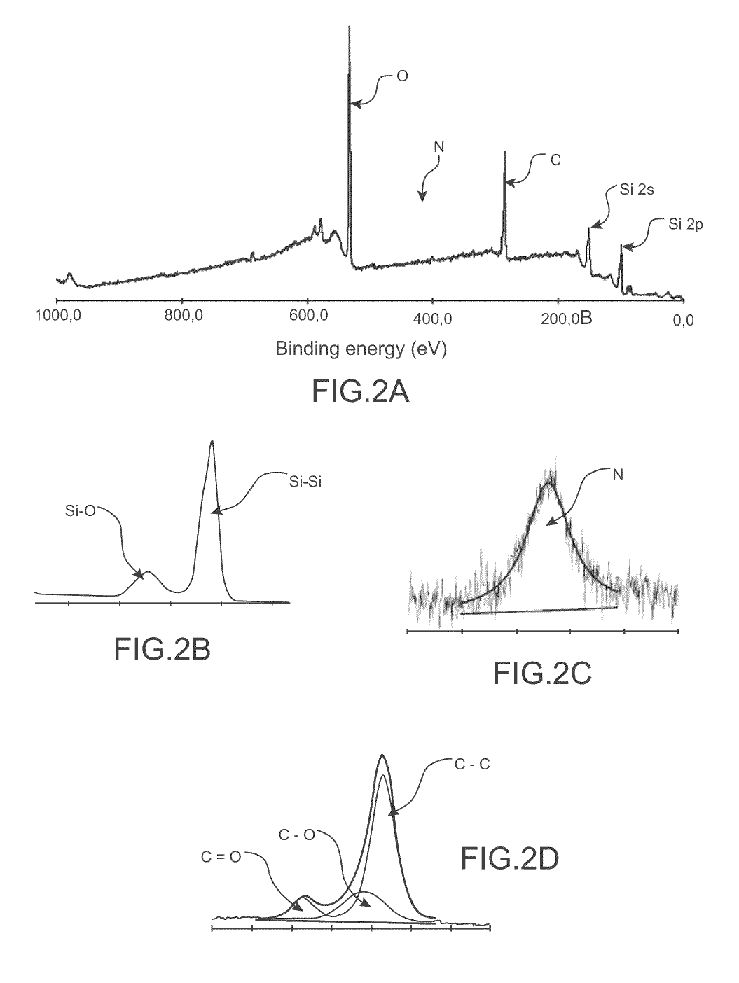

FIG. 2A gives the X-ray photoelectron Spectrophotometry spectrum (XPS) of the molecule 2 according to the invention, grafted on a hydrogenated silicon (100) substrate, and FIGS. 2B-2D are enlargements of portions of the spectrum of FIG. 2A.

In FIG. 2A, the peaks corresponding to O, N, C, Si (2s), and Si (2p) have been indicated.

In FIG. 2B, the peaks corresponding to Si—O and Si—Si bonds have been identified.

In FIG. 2C, the peak corresponding to the nitrogen atoms of molecule 2 has been identified.

In FIG. 2C, the peaks corresponding to the C═O, C—N and C—C bonds of molecule 2 have been identified.

### Example 5

### Measurement of the Electric Properties of the Molecules 1, 2 and 3 Grafted on Silicon

In order to measure the electric properties of the molecules 1, 2 and 3 according to the invention grafted on silicon, electrochemical capacitors were then made on a silicon substrate. (Type P conductor, 7-10 Ω·cm doped with boron).

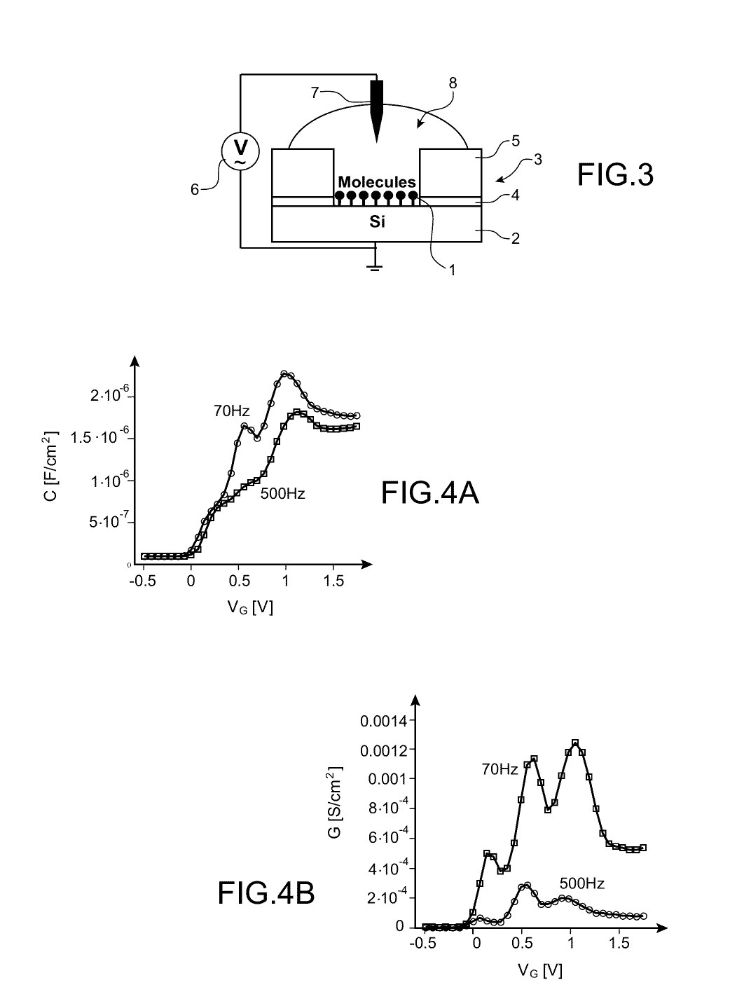

The measurement device is illustrated in FIG. 3.

The active surface area of the capacitance (150×300 μm²) comprising the molecules according to the invention (1) grafted on the silicon (2), is built at the center of thin walls (3) of SiO₂ consisting over a thickness of 500 nm of thermal oxide SiO₂ (4) and then over a thickness of 10 μm of SiO₂ which was grown by PECVD (5).

Sacrificial oxide SiO₂ is grown by PECVD over a thickness of 10 nm on the active silicon surface area, and this oxide is removed by pickling by immersion in a 1% HF aqueous solution for one minute, and then by drying under argon just before grafting the molecules (1) according to the invention. It is then proceeded with hydrogenation under the conditions already specified above.

The grafting of the molecules (1) is carried out as this has already been described above, by a thermal hydrosilylation reaction by placing the freshly pickled silicon substrate in a solution of mesitylene containing the molecule and then by refluxing for 2 hours with heating. The reaction should be conducted under an inert atmosphere.

The electric properties of the molecules on Si are investigated by means of Capacitance-Voltage (C-V) and Conductance-Voltage (G-V) measurements.

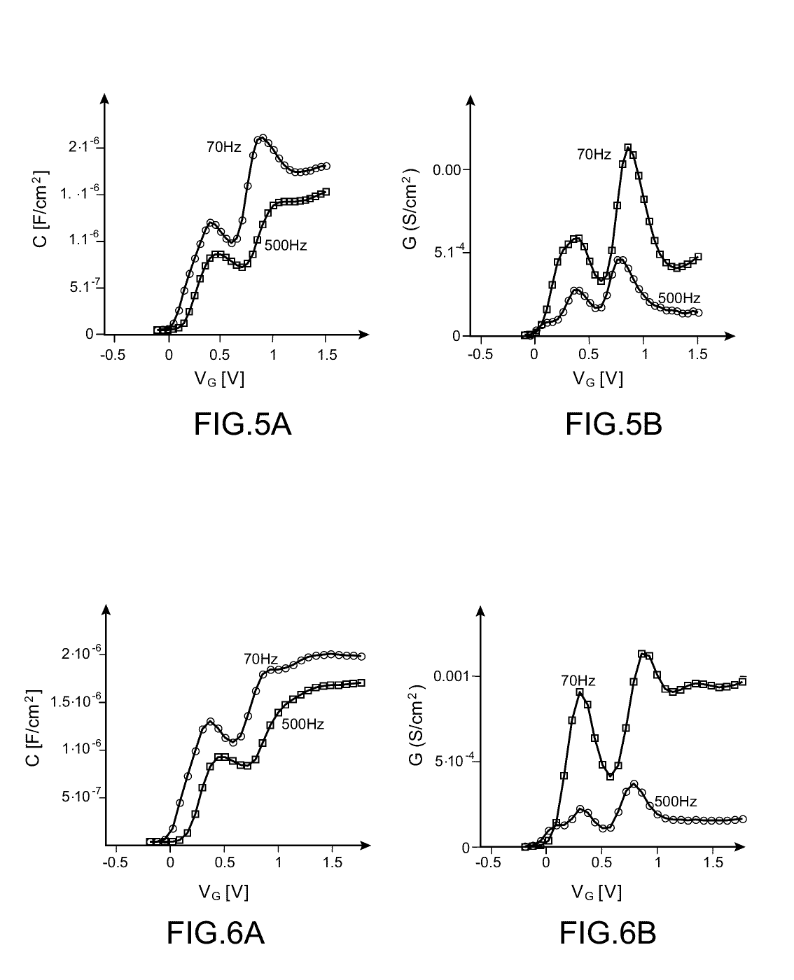

The measurements were conducted by using a Agilent® 4284 A potentiometer (6) in an inert atmosphere (nitrogen). The gate voltage (VG) is applied by means of a silver electrode (7). During the electric characterizations, a drop of electrolyte (8) (a 1.0 M solution of tetrabutylammonium hexafluorophosphate in propylene carbonate) is used as a conducting contact gate with the monolayer of molecules (1). The silver electrode (7) is soaked in the electrolyte drop (8). The results of the measurements of the electric properties of the molecules 1, 2 and 3 according to the invention grafted on silicon are respectively plotted on the graphs of FIGS. 4A and 4B (molecule 1), of FIGS. 5A and 5B (molecule 2), and of FIGS. 6A and 6B (molecule 3).

Table 2 below gives the potential (in V) corresponding to the occurrence of redox peaks for each of the molecules according to the invention prepared in the Examples 1 to 3, grafted on silicon.

TABLE 2
Potential for occurrence of the redox peaks (V)

| Molecule | Peak 1 | Peak 2 |
|---|---|---|
| Without any chain | ~0.5-0.6 V | ~0.9-1.1 V |
| (R = H) | | |
| R = C₈ chain | ~0.3-0.4 V | ~0.75-0.85 V |
| R = C₁₂ chain | ~0.3-0.4 V | ~0.75-0.85 V |

The capacitance and conductance curves (C-V and G-V) show the presence of two peaks, characteristic of the responses of the two redox states of the molecules.

These results confirm a reversible transfer of charges between the silicon and the pi-conjugate core of the molecules according to two distinct charge states with 1 or 2 electrons per molecule.

Table 2 clearly shows that by adding a C₈ or C₁₂ chain for example, it is possible to modulate the charge transfer potentials between the pi-conjugate core and the silicon as compared with the case without any chain, thereby further reducing the potentials.

1. An organic redox active compound with reversible storage of charges, the compound characterized by a formula (I):
R-M-Y-T (I)
wherein:
R represents a deconjugating group;
M represents an organic redox active fragment, not comprising any metal ion or metal, capable of reversibly storing at least one charge;
T represents a tripod group comprising three groups F, bound to a same atom, and capable of being chemically grafted to a surface of a solid substrate; and
Y represents a spacer group separating the redox active fragment M from the tripod group T.

2. The compound according to claim 1, wherein the substrate is made of an insulating material, of a semiconducting material, of a metal material, or of a combination thereof.

3. The compound according to claim 1 wherein the group R is selected from the group consisting of hydrogen, alkyl groups, alkoxy groups, heterocycles, and aryl groups.

4. The compound according to claim 1 wherein the organic redox active fragment M is selected from fragments comprising one or more polycyclic group(s) with pi conjugation.

5. The compounds according to claim 4, wherein the redox active fragment M fits the following formulas (IIA), (IIB), (IIC), (IID) or (IIE): 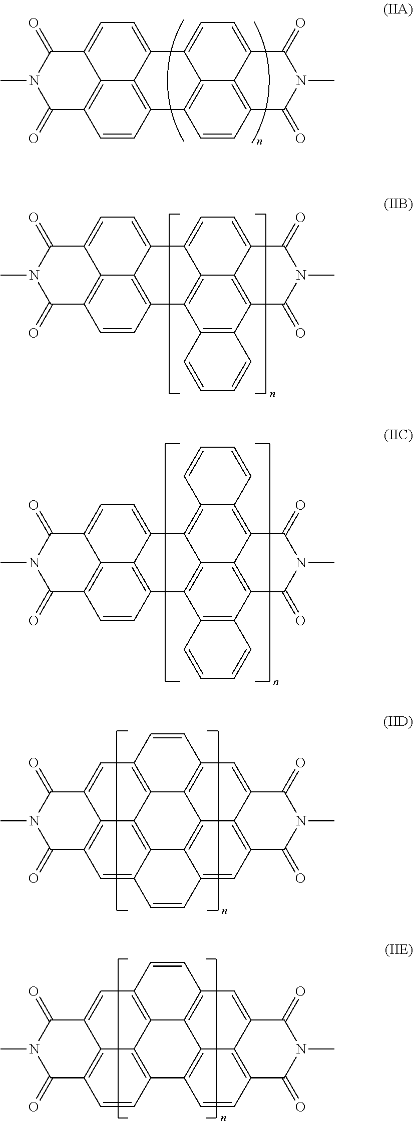
wherein n is an integer between 0 to 6.

6. The compound according to claim 1 wherein the fragment M is further substituted with one or more electron donor or electron acceptor group(s).

7. The compound according to claim 1, wherein the spacer group Y is selected from divalent alkyl groups, divalent alkoxy groups, divalent heterocycles, and divalent aryl groups.

8. The compound according to claim 1 wherein the groups F capable of being chemically bound to a surface of a solid substrate are selected from the group consisting of: hydroxyl, mercapto, selenyl, telluryl, cyano, isocyano, carboxyl, amino, dihydroxyphosphoryl, dithio, dithiocarboxyl, diazonium, halogeno, alkenyl such as allyl, alkynyl, alkoxyl, silyl, phosphate-phosphonate; and carbon chains including one or more atoms selected from the groups consisting of: hydroxyl, mercapto, selenyl, telluryl, cyano, isocyano, carboxyl, amino, dihydroxyphosphoryl, dithio, dithiocarboxyl, diazonium, halogeno, alkenyl.

9. The compound according to claim 8 wherein the tripod group comprises a 4-allylhepta-1,6-dien-4-yl group of formula —C—(—CH₂—CH═CH₂)₃ or a group of formula —Si—(—CH₂—CH═CH₂)₃.

10. The compound according to claim 1 which fits the following formula (IIIA), (IIIB), (IIIC), (IIID) or (IIIE): 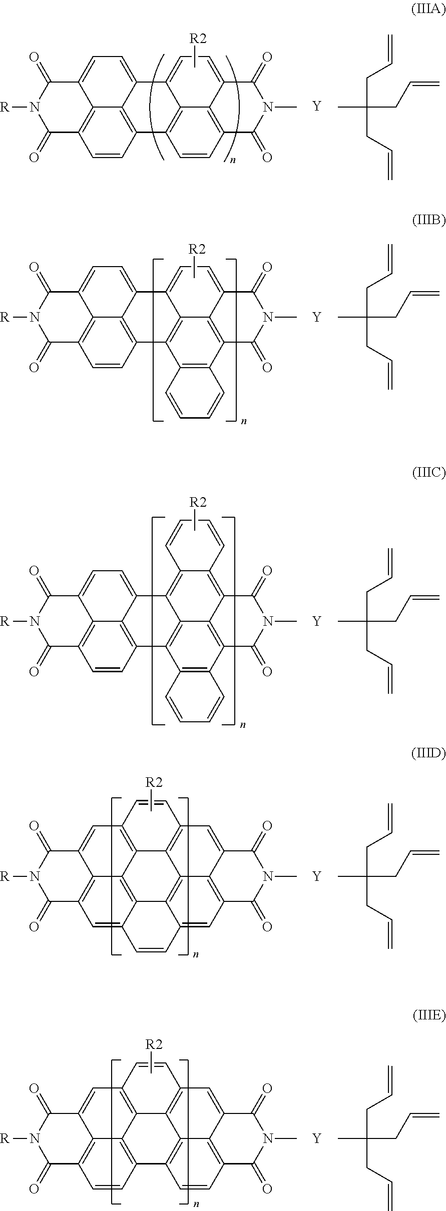
wherein R and Y are as defined in claim 1, n is an integer including all the values from 0 to 6, and R2 represents a hydrogen, an electron donor, or electron attractor group.

11. The compound according to claim 9 comprising:
N-[4-(4-allylhepta-1,6-dien-4-yl)phenyl]-1,4,5,8-naphthalenetetracarboxydiimide;
N-[4-(4-allylhepta-1,6-dien-4-yl)phenyl]-N′-n-octyl-1,4,5,8 naphthalenetetracarboxy-diimide; or
N-[4-(4-allylhepta-1,6-dien-4-yl)phenyl]-N′-n-dodecyl-1,4,5,8-naphthalenetetracarboxy-diimide.

12. A substrate having a surface on which organic redox active compounds are chemically grafted, wherein the organic redox active compounds are capable of storing charges according to claim 1.

13. The substrate according to claim 12 wherein the semiconducting material is silicon, or doped silicon.

14. The substrate according to claim 12, wherein the organic redox active compounds are capable of storing charges form a monolayer at the surface of the substrate, for example with a thickness from about 2 to about 3 nm.

15. A molecular memory device comprising a redox active compound capable of storage of charges as defined in claim 1.

16. An electronic apparatus comprising at least one molecular memory device, wherein the molecular memory device is defined according to claim 15.

17. An electronic apparatus according to claim 16 which is selected from the group consisting of portable electronic apparatuses, digital still cameras, cellphones, printers, portable computers, and sound playing and recording devices.

18. A method of using organic redox active compounds capable of storing charges according to claim 1, the method comprising using the compounds as molecules capable of storing charges in a molecular memory device.

19. A molecular memory device comprising a redox active compound capable of storage of charge as defined in claim 12.

20. An electronic apparatus comprising at least one molecular memory device according to claim 19.

21. The compound according to claim 1, wherein the tripod group is covalently grafted to the substrate.

22. The compound according to claim 8, wherein the carbon chain includes 1 to 5 carbon atoms.

23. The compound according to claim 8, wherein the carbon chain includes 3 carbon atoms.
